(19) United States
(12) Patent Application Publication
(10) Pub. No.: US 2024/0389912 A1
(43) Pub. Date: Nov. 28, 2024

(54) ADJUSTABLE BOOTY TO HEAT AN INFANT'S HEEL PRIOR TO SAMPLING

(71) Applicant: Alexis Kwayisi, Elgin, SC (US)

(72) Inventor: Alexis Kwayisi, Elgin, SC (US)

(21) Appl. No.: 18/667,029

(22) Filed: May 17, 2024

Related U.S. Application Data
(60) Provisional application No. 63/468,557, filed on May 24, 2023.

(51) Int. Cl. A61B 5/15; A43B 7/02; A61F 7/00; A61F 7/02

(52) U.S. Cl. CPC A61B 5/150076; A43B 7/02; A61B 5/150022; A61F 7/02; A61B 2503/045; A61F 2007/0045; A61F 2007/0228; A61F 2007/0236

(57) ABSTRACT

A booty for an infant that heats the heel of the infant while preventing potential bruising, infection or cartilage damage caused by frequent heel sticks from blood tests being taken at the heel of the infant. The booty has a flexible body with a closure opening to allow the booty to be placed on the foot of the infant. The booty also has a closure device to adjust the size of the closure opening and further secure the booty to the foot of the infant. A warming device is included to heat the heel of the infant. The booty includes an attachment device to securely attach the warming device to the booty.

### CLAIM TO PRIORITY

This utility application is based on provisional application No. 63/468,557 filed May 24, 2023, entitled “ADJUSTABLE BOOTY TO HEAT AN INFANT'S HEEL PRIOR TO SAMPLING” the contents of which are expressly incorporated herein by reference.

### FIELD OF INVENTION

The present disclosure relates to devices and methods used to improve the quality of blood samples obtained from an infant. In particular, a booty is provided to proximately warm the heel of the infant from which a blood sample may be taken.

### BACKGROUND

Neonatal research is a method of early diagnosis of diseases that especially applies to newborn children between about twenty-four to forty-eight hours or so after being born.

Neonatal screening is a method of early diagnosis of many diseases, such as phenylketonuria or hyperphenylalaninemia, adrenal hyperplasia, hypothyroidism, which can cause in the newborn mental retardation, among others, but if detected and treated early and at the appropriate time would not lead to major problems. As research progresses, it is discovered that other diseases may be detected early using this diagnosis method.

Methods of sampling blood from infants include heel puncture, venipuncture, and arterial puncture. Venipuncture involves aspirating blood through a needle from a peripheral vein. Heel puncture involves lancing the lateral aspect of the infant's heel, squeezing the heel or applying pressure to the heel in some way, and collecting the pooled capillary blood. Arterial puncture (usually called a radial artery puncture) is a procedure performed by accessing the arterial system by direct penetration of a peripheral artery, with a needle or catheter.

The neonatal screening method typically involves a heel puncture of a newborn from where samples of blood are extracted using a lancet, for example. When sampling using this method, it is necessary for the blood to fluidly be removed from the infant's body. However, often a lancet does not provide the security that the blood is being fluidly removed from the infant's body. In an effort to overcome this deficiency, the person taking the sample is instructed to rub or massage the newborn's heel prior to puncturing the heel. Often, the person taking the sample may repeat puncturing several times to fluidly obtain a sample. Heel punctures are estimated to be 61-87% of the invasive procedures performed on infants suffering from an illness.

The newborn's heel is delicate and the pressure from the puncture may cause rupture of capillaries, hemorrhage, rupture of cartilage, contamination of the blood sample with interstitial and intracellular fluids. Additionally, rubbing or massaging the heel may cause hemolysis and other injuries that may compromise the quality of samples to be subsequently taken, in case there is a need to repeat the heel sampling. Multi-puncture may be deemed a cruel procedure resisted by the newborn and his/her relatives, making the work performed by the person taking the sample more difficult.

Pain caused by blood sampling is associated with acute behavioral and physiological deterioration. Experience of pain during heel puncture seems to heighten pain responses during subsequent blood sampling from infants. Other adverse effects of blood sampling include bleeding, bruising, hematoma, and infection.

It has been found that warming up the skin in the heel area to be punctured increases the capillary action by vessel dilation or vasodilation. Warming up the skin in this location increases blood flow in the area that is punctured. Academic work and evidence-based practice promotes the need to warm the infant's heel to facilitate obtaining the blood sample. Bandage type patches or disposable cloths are used to warm the child's heels prior to the puncture. These bandages or disposable cloths can be heated or constructed of thermal materials and applied to the area that needs to be heated. Other devices have been constructed to wrap around the feet or ankles of the infant. These devices may also include some form of heating means. If these bandages or cloths are overly heated, they can cause burns to the delicate skin of the babies. Some of the devices may prove to be uncomfortable or annoying to the child who may choose to attempt remove them. This further complicates the work of the person taking the sample and may make it necessary to perform multi-puncture, since proper capillary dilation in the infant's foot cannot otherwise be achieved to gain a good quality sample.

There are other known heaters, including fragments or sectors, that become joined to each other by tape or other attachment elements that are placed around the foot or the heel of the infant. These devices require brooches that engage into holes to avoid allowing the infant from easily removing them. These devices have been shown not to be suitable for babies since they are uncomfortable and the babies may attempt to remove them. The result is the same as in the case of bandages or cloth requiring multi-puncture to achieve the quality blood sample required by the screening methods. Furthermore, the complications introduced by devices increases the production cost that may more readily subject them to reuse ultimately subjecting them to contamination with fluids of other infants.

There remains a need in the art for devices or methods that may be used to obtain a blood sample that is fluidly removed from the body of an infant. In particular, there is a need for a booty that can be used at the heel of an infant allowing the infant's heel to be heated prior to capillary puncture. Such a booty needs to be such that it can be securely attached to surround the heel of the infant but without subjecting the infant to discomfort. Indeed such a device should naturally conform to the infant's foot whereby the device is readily adaptable to be comfortable for the infant but yet allow the heel to become heated for proper sampling.

### SUMMARY OF INVENTION

The present invention relates to a heel wrap to improve the quality of blood samples taken from infants. The present invention also relates to methods of improving the quality of blood samples taken from infants. Without intending to be bound by theory, the heel wrap of the invention. Certain exemplary polymeric compounds are further included in this disclosure.

An aspect of the invention provides a booty having a flexible body with a closure opening to allow the booty to be place on the foot of the infant, a closure device to adjust the size of the closure opening and secure the booty to the foot of an infant, a warming device to heat a heel of the infant, and an attachment device to attach the warming device to the booty. According to an embodiment of the invention, the flexible body includes one or more of a nonwoven material or a woven material. In another embodiment of the invention, the closure device includes two straps.

In an embodiment of the invention, the closure opening is a v-shape opening beginning at the top of the booty where the booty is first introduced to the foot of the infant and continuing along the booty to close at a location in an area of where an ankle of the infant begins. In certain embodiments of the invention, the closure device includes at least one strap to hold the closure opening securely when the booty is fitted on the infant. Further pursuant to this embodiment of the invention, the strap may have any one or more of an elastic band, an adhesive strip having an outer layer that upon removal allows the strap to be secured to the booty, a Velcro material that is configured to be secured to the body of the booty, and laces that may be tied off at a tie-down loop provided on the booty.

In an embodiment of the invention, the attachment device comprises a conforming material to allow for various sizes of the warming device to be attached to the booty. In certain embodiments of the invention the attachment device is a sleeve that holds the warming device that is inserted into the booty. Further pursuant to this embodiment of the invention, the warming device proximately heats the heel of an infant.

In another embodiment of the invention, the warming device attaches to the booty such that it is substantially adjacent to the heel of the infant.

In another aspect, the invention provides a method for heating a heel of an infant including the following steps: providing a booty having a warming device, securing the booty to the foot of the infant to at least surround the heel of the infant, and heating the heel of the infant.

An embodiment of the invention includes additionally sampling blood from the heel of the infant.

Other aspects and embodiments will become apparent upon review of the following description taken in conjunction with the accompanying drawings. The invention, though, is pointed out with particularity by the included claims.

### BRIEF DESCRIPTION OF THE DRAWINGS

Having thus described the invention in general terms, reference will now be made to the accompanying drawings, which are not necessarily drawn to scale, and wherein:

FIG. 1 is a front view of a booty of the invention having one closure strap;

FIG. 2 is a right-side view of a booty of the invention having one closure strap;

FIG. 3 is a left-side view of a booty of the invention having one closure strap;

FIG. 4 is a front view of a booty of the invention having one closure strap, the booty configured to proximately heat the heel of an infant;

FIG. 5 is a right-side view of a booty of the invention having one closure strap, the booty configured to proximately heat the heel of an infant;

FIG. 6 is a left-side view of a booty of the invention having one closure strap, the booty configured to proximately heat the heel of an infant;

FIG. 7 is a front view of a booty of the invention having two closure straps;

FIG. 8 is a right-side view of a booty of the invention having two closure straps;

FIG. 9 is a left-side view of a booty of the invention having two closure straps;

FIG. 10 is a front view of a booty of the invention having two closure straps, the booty configured to proximately heat the heel of an infant;

FIG. 11 is a right-side view of a booty of the invention having two closure straps, the booty configured to proximately heat the heel of an infant; and

FIG. 12 is a left-side view of a booty of the invention having two closure straps, the booty configured to proximately heat the heel of an infant.

### DETAILED DESCRIPTION OF THE INVENTION

The present invention now will be described more fully hereinafter with reference to the accompanying drawings, in which some, but not all embodiments of the inventions are shown. Preferred embodiments of the invention may be described, but this invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments of the invention are not to be interpreted in any way as limiting the invention.

As used in the specification and in the appended claims, the singular forms “a”, “an”, and “the” include plural referents unless the context clearly indicates otherwise. For example, reference to “a strap” may include a plurality of such straps.

It will be understood that relative terms may be used herein to describe one element's relationship to another element as, for example, may be illustrated in the Figures. It will be understood that relative terms are intended to encompass different orientations of the elements in addition to the orientation of elements as illustrated in the Figures. It will be understood that such terms can be used to describe the relative positions of the element or elements of the invention and are not intended, unless the context clearly indicates otherwise, to be limiting.

Embodiments of the present invention are described herein with reference to various perspectives, including, for example, perspective views that are representations of idealized embodiments of the present invention. As a person having ordinary skill in the art would appreciate, variations from or modifications to the shapes as illustrated in the Figures or the described perspectives are to be expected in practicing the invention. Such variations and/or modifications can be the result of manufacturing techniques, design considerations, and the like, and such variations are intended to be included herein within the scope of the present invention and as further set forth in the claims that follow. The articles of the present invention and their respective components described or illustrated in the Figures are not intended to reflect a precise description or shape of the component of an article and are not intended to limit the scope of the present invention.

Although specific terms are employed herein, they are used in a generic and a descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

As used herein, a “booty” means a device designed to fit surrounding, at least in part, the foot and ankle of an infant. More particular to the booty as claimed herein, without intending to be bound by theory, the booty is designed and adaptable to fit comfortably on the foot of the infant. Moreover, as further disclosed herein, the booty includes an insert to allow a warming device to be inserted and heat the foot, and, more specifically, with reference to certain embodiments of the invention, the heel of the infant to allow for a specimen of blood to be taken from the infant.

As used herein, the term “flexible body” means a body that is constructed in such a way and with the necessary material or materials to allow the flexible body to be easily adapted to surround the foot and/or the ankle of the infant. Without intending to be limiting, a flexible body may include a material such as various soft plastics, non-fusible yarns, textiles, or a fabric that is capable of flexing and/or being displaced with relatively little force. Suitable flexible materials allow expansion of any underlying sections of the flexible body. In an embodiment of the invention, the flexible body comprises any one of a nonwoven material and a woven material. In certain embodiments of the invention, the nonwoven material and/or the woven material is an interface fabric. A “flexible” material will bend or deform more readily under such forces than a “rigid” material will bend or deform under comparable forces.

As used herein, an “infant” is intended to include a newborn, a baby, or any child that is of the age from which blood sampling from the heel, as described herein, is required.

As used herein, the terms “strap” and “closure strap” are intended to be interpreted as a device that allows for the closure opening of the booty, in preferred embodiments substantially surrounding the ankle of the infant, to be closed to allow the booty to be fitted about the foot of the infant. In certain embodiments of the invention, the strap may be constructed of an elastic band, an adhesive strip having an outer layer that upon removal allows the strap to be secured to the booty to allow the booty to remain in place surrounding the foot of the infant. In other embodiments of the invention, the strap may be constructed of a Velcro material that is configured to be secured to the body of the booty. In yet other embodiments of the invention, the strap comprises to laces that may be tied off at a tie-down loop provided on the booty. A strap may be constructed of any securing device and/or mechanism that is known in the art.

An aspect of the invention provides a booty having a flexible body that is capable of being placed upon and adapted to fit the foot and ankle of an infant. In certain embodiments of the invention, the flexible body has a closure opening to allow the booty to be easily be placed through the foot of the infant when in the open position and, in some embodiments of the invention, to surround a portion of the ankle of the infant. In certain embodiments of the invention, the closure opening is a v-shape opening beginning at the top of the booty where it is first introduced to the foot of the infant and continuing along the booty to close at a location in the area of where the ankle of the infant begins.

The inventor has conceived of a booty design that allows the booty to be securely and comfortably be placed around the heel of the infant, warming the heel of the infant, and allowing for a blood sample to be taken from the warmed heel of the infant.

The booty further comprises a closure device to allow the closure opening to be closed to surround that portion of the infant that is otherwise exposed. In certain embodiments of the invention, the closure device comprises at least one strap.

The booty additionally comprises an attachment device to allow a warming device to be attached to the booty and placed adjacent to the foot, more preferably, adjacent to the heel, to allow the adjacent surface of the infant to become heated. Without intending to be bound by theory, the warming device may minimize the potential bruising, infection or cartilage damage caused by frequent heel sticks from blood tests being taken at the heel of the infant. In an embodiment of the invention, the warming device comprises sodium acetate and water. In another embodiment of the invention, the warming device comprises of sodium thiosulfate.

The attachment device may be adapted to hold any size or orientation of a warming device that is available for such a use. In an embodiment of the invention, the attachment device comprises a conforming material to allow for various sizes of the warming device to be included in the attachment device.

In one embodiment of the invention, the attachment device is a sleeve that allows the warming device to be inserted into the booty such that the warming device lies adjacent to the foot of the infant, more preferably, adjacent to the heel of the infant. In an embodiment of the invention, the sleeve is stretchable to allow it to easily adapt to many of the sizes of warming devices that are available.

In certain embodiments of the invention, the booty is configured to proximately heat the heel of the infant. The term proximately is intended to designate that the booty substantially fits perhaps some portion of the arch-side and the backside of the infant's foot at the heel area to allow the warming device to be substantially directed to the heel of the infant.

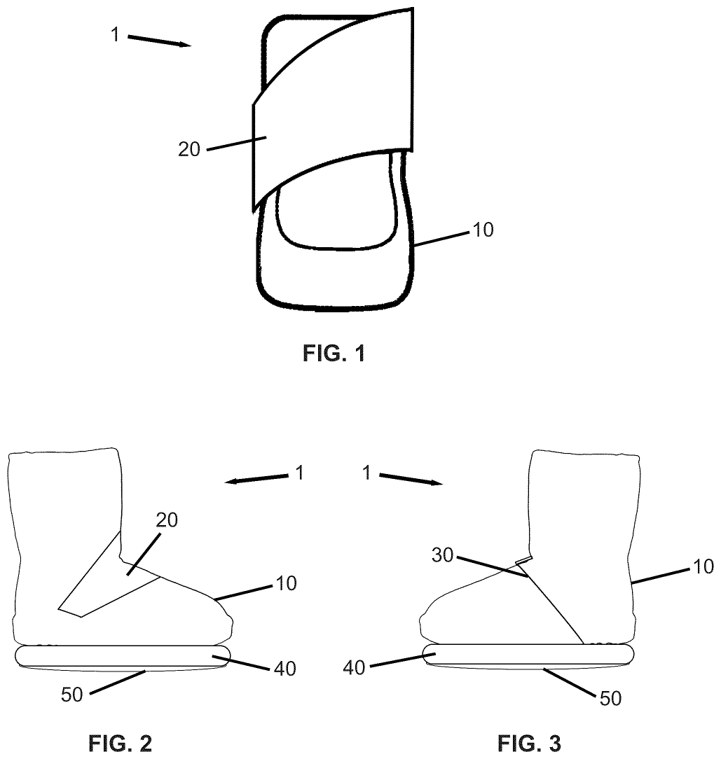

FIG. 1 is a front view of a booty of the invention having one closure strap. The booty 1 comprises a flexible body 10 and a closure strap 20 to allow a closure opening (not visible) of the flexible body 10 to be secured shut allowing the booty 1 to fit comfortably and securely around the foot of an infant.

FIG. 2 is a right-side view of a booty of the invention having one closure strap. The booty 1 comprises a flexible body 10 and the closure strap 20. This view of the booty 1 also shows a warming device 40 included at the base of the flexible body 10 that is secured to the booty 1 with the use of a sleeve 50.

FIG. 3 is a left-side view of a booty of the invention having one closure strap. In addition to the flexible body 10, the warming device 40, and the sleeve 50, this view of the booty 1 shows a strap seam 30 for the closure strap used to secure the booty 1 to the infant.

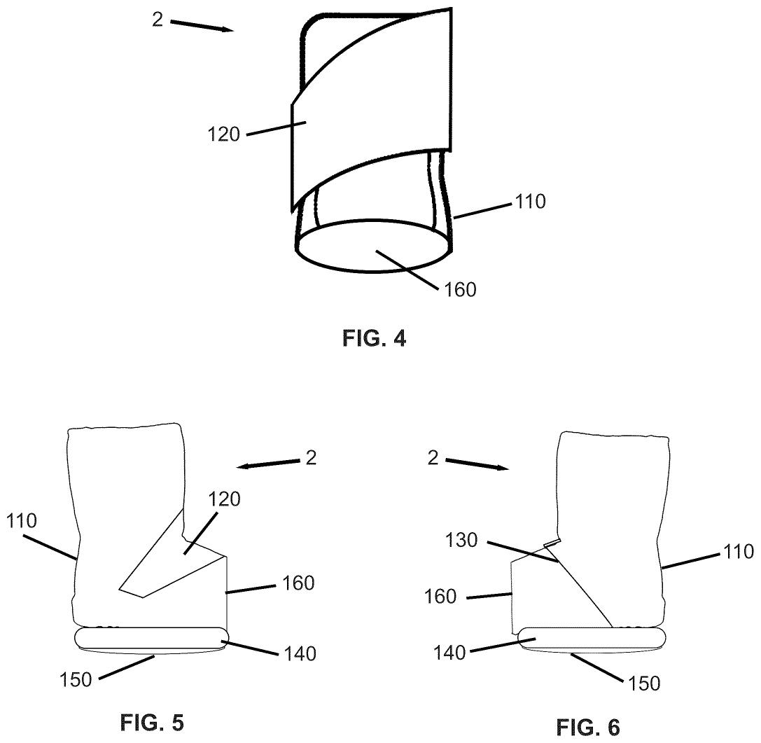

FIG. 4 is a front view of a booty of the invention having one closure strap, the booty configured to proximately heat the heel of an infant. The booty 2 includes a flexible body 110 and a closure strap 120. The front opening 160 allows a front part of the infant's foot to be inserted through the booty 2 to allow a warming device to be positioned substantially at a heel of the infant.

FIG. 5 is a right-side view of a booty of the invention having one closure strap, the booty configured to proximately heat the heel of an infant. While FIG. 5 shows the booty 2 having a flexible body 110 and a closure strap 120, the booty 2 includes a warming device 140, and a sleeve 150. Additionally, the booty 2 is configured to have a front opening 160 to allow the infant's foot to continue to be extended through the booty 2 to allow the warming device 140 to be positioned substantially at a heel of the infant.

FIG. 6 is a left-side view of a booty of the invention having one closure strap, the booty configured to proximately heat the heel of an infant. While FIG. 6 shows the booty 2 having a flexible body 110, a strap seam 130, a warming device 140, and a sleeve 150, the booty 2 is configured to have a front opening 160 to allow the infant's foot to continue to be extended through the booty 2 to allow the warming device 140 to be positioned substantially at a heel of the infant.

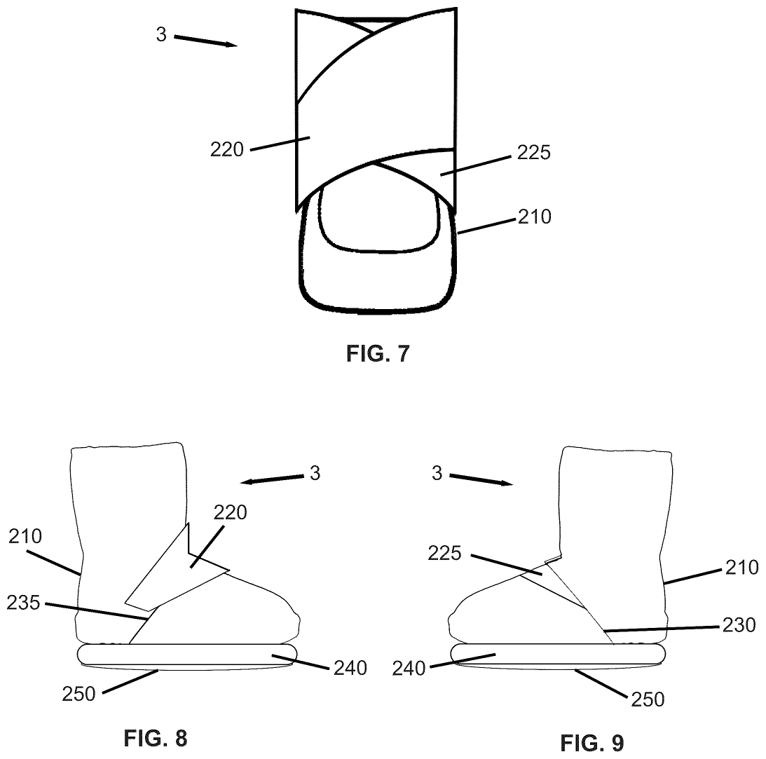

FIG. 7 is a front view of a booty of the invention having two closure straps. The booty 3 comprises a flexible body 210, a right-side closure strap 220 extending to the right-side of the booty 3, and a left-side closure strap 225 extending to the left-side of the booty 3.

FIG. 8 is a right-side view of a booty of the invention having two closure straps. As in FIG. 7, FIG. 8 shows that the booty 3 has a flexible body 210 and a right-side closure strap 220. FIG. 8 also shows the warming device 240 and the sleeve 250 as well as a left-side closure strap seam 235.

FIG. 9 is a left-side view of a booty of the invention having two closure straps. As in FIG. 7, FIG. 9 shows that the booty 3 has a flexible body 210 and a left-side closure strap 225.

FIG. 9 also shows the warming device 240 and the sleeve 250 as well as a right-side closure strap seam 230.

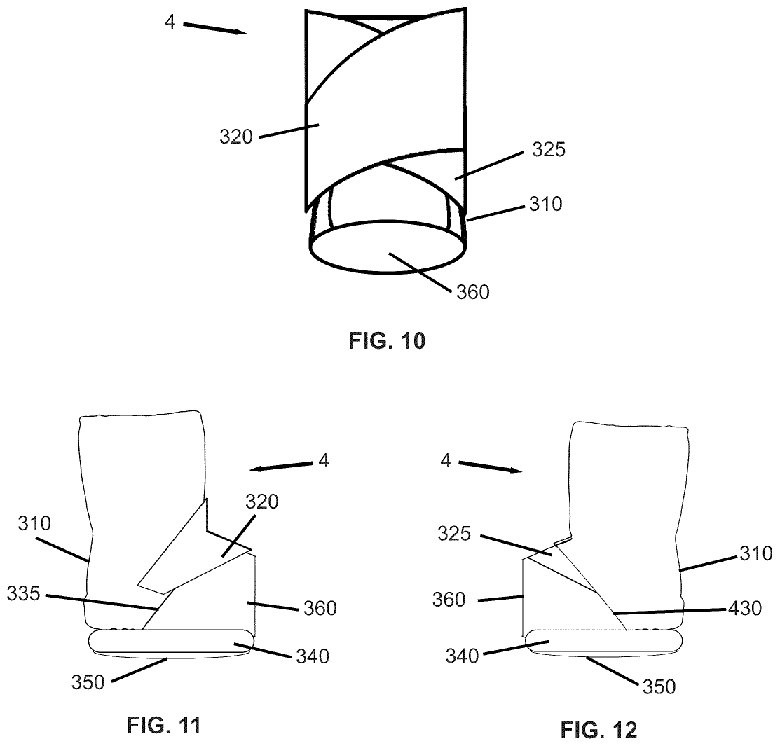

FIG. 10 is a front view of a booty of the invention having two closure straps, the booty configured to proximately heat the heel of an infant. The booty 4 includes a flexible body 310, a right-side closure strap 320, and a left-side closure strap 325. The front opening 360 allows a front part of the infant's foot to be inserted through the booty 4 to allow a warming device to be positioned substantially at a heel of the infant.

FIG. 11 is a right-side view of a booty of the invention having two closure straps, the booty configured to proximately heat the heel of an infant. While FIG. 10 shows the booty 4 having a flexible body 310, a right-side closure strap 320, and a left-side closure strap 325, the booty 4 of FIG. 11 also includes a warming device 340, and a sleeve 350. Additionally, the booty 4 is configured to have a front opening 360 to allow the infant's foot to continue to be extended through the booty 4 to allow the warming device 340 to be positioned substantially at a heel of the infant.

FIG. 12 is a left-side view of a booty of the invention having two closure straps, the booty configured to proximately heat the heel of an infant. While FIG. 10 shows the booty 4 having a flexible body 310, a right-side closure strap 320, and a left-side closure strap 325, the booty 4 of FIG. 12 also includes a warming device 340, and a sleeve 350. Additionally, the booty 4 is configured to have a front opening 360 to allow the infant's foot to continue to be extended through the booty 4 to allow the warming device 340 to be positioned substantially at a heel of the infant.

In an embodiment of the invention, an access opening may be provided in the booty to allow for a blood sample to be taken from the infant while the booty remains in place on the infant's foot. The access point may be secured to the booty by any attachment means known in the art.

Another aspect of the invention provides a method for heating a heel of an infant that includes the steps of providing a booty having a warming device, securing the booty to the foot of the infant to at least surround the heel of the infant, and heating the heel of the infant. Further to this embodiment of the invention, the method comprises the step of sampling blood from the heel of infant.

Having the benefit of the teachings presented in the descriptions herein, many modifications and other embodiments of the invention set forth herein will come to mind to one skilled in the art to which this invention pertains. It will be appreciated by those skilled in the art that changes could be made to the embodiments described herein without departing from the broad inventive concept thereof. Therefore, it is understood that this invention is not limited to the particular embodiments disclosed, but it is intended to cover modifications within the spirit and scope of the present invention as defined by the included claims.

That which is claimed:

1. A booty for an infant comprising
a flexible body having a closure opening to allow the booty to be placed on the foot of the infant;
a closure device to adjust the size of the closure opening and secure the booty to the foot of the infant;
a warming device to heat a heel of the infant; and
an attachment device to attach the warming device to the booty.

2. The booty of claim 1, wherein the flexible body comprising at least one of a nonwoven material and a woven material.

3. The booty of claim 1, wherein the closure opening is a v-shape opening beginning at the top of the booty where the booty is first introduced to the foot of the infant and continuing along the booty to close at a location in an area of where an ankle of the infant begins.

4. The booty of claim 1, wherein the closure device includes at least one of a strap.

5. The booty of claim 4, wherein the strap having any one or more of an elastic band, an adhesive strip having an outer layer that upon removal allows the strap to be secured to the booty, a Velcro material that is configured to be secured to the body of the booty, and laces that may be tied off at a tie-down loop provided on the booty.

6. The booty of claim 1, wherein the attachment device comprises a conforming material to allow for various sizes of the warming device to be attached to the booty.

7. The booty of claim 6, wherein the attachment device is a sleeve that holds the warming device that is inserted into the booty.

8. The booty of claim 1, wherein the warming device attaches to the booty such that it is substantially adjacent to the heel of the infant.

9. The booty of claim 1, wherein the warming device comprises sodium acetate and water.

10. The booty of claim 1, wherein the warming device comprises sodium thiosulfate.

11. The booty of claim 8, wherein the booty includes a front opening through which the foot of the infant may continue to be directed.

12. The booty of claim 11, wherein the warming device proximately heats the heel of an infant.

13. The booty of claim 4, wherein the closure device includes two straps.

14. The booty of claim 1 additionally comprising an access opening to allow for a blood sample to be taken from the infant while the booty remains in place on the infant's foot.

15. A method for heating a heel of an infant comprising the following:
providing a booty having a warming device
securing the booty to the foot of the infant to at least surround the heel of the infant, and
heating the heel of the infant.

16. The method of claim 15, additionally comprising sampling blood from the heel of the infant.
